(19) United States
(12) Patent Application Publication
(10) Pub. No.: US 2014/0287948 A1
(43) Pub. Date: Sep. 25, 2014

(54) BIOMARKERS AND METHODS FOR PREDICTING PRETERM BIRTH

(71) Applicant: Sera Prognostics, Inc., Salt Lake City, UT (US)

(72) Inventors: John Jay BONIFACE, Salt Lake City, UT (US); Gregory Charles CRITCHFIELD, Salt Lake City, UT (US); Durlin Edward HICKOK, Seattle, WA (US)

(73) Assignee: Sera Prognostics, Inc., Salt Lake City, UT (US)

(21) Appl. No.: 14/212,739

(22) Filed: Mar. 14, 2014

Related U.S. Application Data
(60) Provisional application No. 61/798,504, filed on Mar. 15, 2013.

(51) Int. Cl. G01N 33/68

(52) U.S. Cl. CPC G01N 33/689
USPC 506/9; 506/18; 506/12

(57) ABSTRACT

The disclosure provides biomarker panels, methods and kits for determining the probability for preterm birth in a pregnant female. The present disclosure is based, in part, on the discovery that certain proteins and peptides in biological samples obtained from a pregnant female are differentially expressed in pregnant females that have an increased risk of developing in the future or presently suffering from preterm birth relative to matched controls. The present disclosure is further based, in part, on the unexepected discovery that panels combining one or more of these proteins and peptides can be utilized in methods of determining the probability for preterm birth in a pregnant female with relatively high sensitivity and specificity. These proteins and peptides disclosed herein serve as biomarkers for classifying test samples, predicting a probability of preterm birth, monitoring of progress of preterm birth in a pregnant female, either individually or in a panel of biomarkers.

The invention relates generally to the field of personalized medicine and, more specifically to compositions and methods for determining the probability for preterm birth in a pregnant female.

### BACKGROUND

According to the World Heath Organization, an estimated 15 million babies are born preterm (before 37 completed weeks of gestation) every year. In almost all countries with reliable data, preterm birth rates are increasing. See, World Health Organization; March of Dimes; The Partnership for Maternal, Newborn & Child Health; Save the Children, Born too soon: the global action report on preterm birth, ISBN 9789241503433 (2012). An estimated 1 million babies die annually from preterm birth complications. Globally, preterm birth is the leading cause of newborn deaths (babies in the first four weeks of life) and the second leading cause of death after pneumonia in children under five years. Many survivors face a lifetime of disability, including learning disabilities and visual and hearing problems.

Across 184 countries with reliable data, the rate of preterm birth ranges from 5% to 18% of babies born. Blencowe et al., “National, regional and worldwide estimates of preterm birth.” The Lancet, 9; 379(9832):2162-72 (2012). While over 60% of preterm births occur in Africa and south Asia, preterm birth is nevertheless a global problem. Countries with the highest numbers include Brazil, India, Nigeria and the United States of America. Of the 11 countries with preterm birth rates over 15%, all but two are in sub-Saharan Africa. In the poorest countries, on average, 12% of babies are born too soon compared with 9% in higher-income countries. Within countries, poorer families are at higher risk. More than three-quarters of premature babies can be saved with feasible, cost-effective care, for example, antenatal steroid injections given to pregnant women at risk of preterm labour to strengthen the babies' lungs.

Infants born preterm are at greater risk than infants born at term for mortality and a variety of health and developmental problems. Complications include acute respiratory, gastrointestinal, immunologic, central nervous system, hearing, and vision problems, as well as longer-term motor, cognitive, visual, hearing, behavioral, social-emotional, health, and growth problems. The birth of a preterm infant can also bring considerable emotional and economic costs to families and have implications for public-sector services, such as health insurance, educational, and other social support systems. The greatest risk of mortality and morbidity is for those infants born at the earliest gestational ages. However, those infants born nearer to term represent the greatest number of infants born preterm and also experience more complications than infants born at term.

To prevent preterm birth in women who are less than 24 weeks pregnant with a history of early premature birth and an ultrasound showing cervical opening, a surgical procedure known as cervical cerclage can be employed in which the cervix is stitched closed with strong sutures. For women less than 34 weeks pregnant and in active preterm labor, hospitalization may be necessary as well as the administration of medications to temporarily halt preterm labor an/or promote the fetal lung development. If a pregnant women is determined to be at risk for preterm birth, health care providers can implement various clinical strategies that may include preventive medications, for example, hydroxyprogesterone caproate (Makena) injections and/or vaginal progesterone gel, restrictions on sexual activity and/or other physical activities, and alterations of treatments for chronic conditions, such as diabetes and high blood pressure, that increase the risk of preterm labor.

There is a great need to identify and provide women at risk for preterm birth with proper antenatal care. Women identified as high-risk can be scheduled for more intensive antenatal surveillance and prophylactic interventions. Current strategies for risk assessment are based on the obstetric and medical history and clinical examination, but these strategies are only able to identify a small percentage of women who are at risk for preterm delivery. Reliable early identification of risk for preterm birth would enable planning appropriate monitoring and clinical management to prevent preterm delivery. Such monitoring and management might include: more frequent prenatal care visits, serial cervical length measurements, enhanced education regarding signs and symptoms of early preterm labor, lifestyle interventions for modifiable risk behaviors and progesterone treatment. Finally, reliable antenatal identification of risk for preterm birth also is crucial to cost-effective allocation of monitoring resources.

The present invention addresses this need by providing compositions and methods for determining whether a pregnant woman is at risk for preterm birth. Related advantages are provided as well.

### SUMMARY

The present invention provides compositions and methods for predicting the probability of preterm birth in a pregnant female.

In one aspect, the invention provides a panel of isolated biomarkers comprising N of the biomarkers listed in Tables 1, 2, 3, 4, 6 and 7. In some embodiments, N is a number selected from the group consisting of 2 to 24. In additional embodiments, the biomarker panel comprises at least two of the isolated biomarkers selected from the group consisting of AFTECCVVASQLR, ELLESYIDGR, and ITLPDFTGDLR.

In some embodiments, the invention provides a biomarker panel comprising at least two of the isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G).

In other embodiments, the invention provides a biomarker panel comprising lipopolysaccharide-binding protein (LBP), prothrombin (THRB), complement component C5 (C5 or CO5), plasminogen (PLMN), complement component C8 gamma chain (C8G or CO8G), complement component 1, q subcomponent, B chain (C1QB), fibrinogen beta chain (FIBB or FIB), C-reactive protein (CRP), inter-alpha-trypsin inhibitor heavy chain H4 (ITIH4), chorionic somatomammotropin hormone (CSH), and angiotensinogen (ANG or ANGT).

Also provided by the invention is a method of determining probability for preterm birth in a pregnant female comprising detecting a measurable feature of each of N biomarkers selected from the biomarkers listed in Tables 1, 2, 3, 4, 6 and 7 in a biological sample obtained from the pregnant female, and analyzing the measurable feature to determine the probability for preterm birth in the pregnant female. In some embodiments, a measurable feature comprises fragments or derivatives of each of the N biomarkers selected from the biomarkers listed in Tables 1, 2, 3, 4, 6 and 7. In some embodiments of the disclosed methods detecting a measurable feature comprises quantifying an amount of each of N biomarkers selected from the biomarkers listed in Tables 1, 2, 3, 4, 6 and 7, combinations or portions and/or derivatives thereof in a biological sample obtained from the pregnant female. In additional embodiments, the disclosed methods of determining probability for preterm birth in a pregnant female further encompass detecting a measurable feature for one or more risk indicia associated with preterm birth.

In some embodiments, the disclosed methods of determining probability for preterm birth in a pregnant female comprises detecting a measurable feature of each of N biomarkers, wherein N is selected from the group consisting of 2 to 24. In further embodiments, the disclosed methods of determining probability for preterm birth in a pregnant female comprises detecting a measurable feature of each of at least two isolated biomarkers selected from the group consisting of AFTECCVVASQLR, ELLESYIDGR, and ITLPDFTGDLR.

In other embodiments, the disclosed methods of determining probability for preterm birth in a pregnant female comprise detecting a measurable feature of each of at least two isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G).

In further embodiments, the disclosed methods of determining probability for preterm birth in a pregnant female comprise detecting a measurable feature of each of at least two isolated biomarkers selected from the group consisting of lipopolysaccharide-binding protein (LBP), prothrombin (THRB), complement component C5 (C5 or CO5), plasminogen (PLMN), complement component C8 gamma chain (C8G or CO8G), complement component 1, q subcomponent, B chain (C1QB), fibrinogen beta chain (FIBB or FIB), C-reactive protein (CRP), inter-alpha-trypsin inhibitor heavy chain H4 (ITIH4), chorionic somatomammotropin hormone (CSH), and angiotensinogen (ANG or ANGT).

In some embodiments of the methods of determining probability for preterm birth in a pregnant female, the probability for preterm birth in the pregnant female is calculated based on the quantified amount of each of N biomarkers selected from the biomarkers listed in Tables 1, 2, 3, 4, 6 and 7. In some embodiments, the disclosed methods for determining the probability of preterm birth encompass detecting and/or quantifying one or more biomarkers using mass sprectrometry, a capture agent or a combination thereof.

In some embodiments, the disclosed methods of determining probability for preterm birth in a pregnant female encompass an initial step of providing a biomarker panel comprising N of the biomarkers listed in Tables 1, 2, 3, 4, 6 and 7. In additional embodiments, the disclosed methods of determining probability for preterm birth in a pregnant female encompass an initial step of providing a biological sample from the pregnant female.

In some embodiments, the disclosed methods of determining probability for preterm birth in a pregnant female encompass communicating the probability to a health care provider. In additional embodiments, the communication informs a subsequent treatment decision for the pregnant female. In further embodiments, the treatment decision comprises one or more selected from the group of consisting of more frequent prenatal care visits, serial cervical length measurements, enhanced education regarding signs and symptoms of early preterm labor, lifestyle interventions for modifiable risk behaviors and progesterone treatment.

In further embodiments, the disclosed methods of determining probability for preterm birth in a pregnant female encompass analyzing the measurable feature of one or more isolated biomarkers using a predictive model. In some embodiments of the disclosed methods, a measurable feature of one or more isolated biomarkers is compared with a reference feature.

In additional embodiments, the disclosed methods of determining probability for preterm birth in a pregnant female encompass using one or more analyses selected from a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, and a combination thereof. In one embodiment, the disclosed methods of determining probability for preterm birth in a pregnant female encompasses logistic regression.

In some embodiments, the invention provides a method of determining probability for preterm birth in a pregnant female encompasses quantifying in a biological sample obtained from the pregnant female an amount of each of N biomarkers selected from the biomarkers listed in Tables 1, 2, 3, 4, 6 and 7; multiplying the amount by a predetermined coefficient, and determining the probability for preterm birth in the pregnant female comprising adding the individual products to obtain a total risk score that corresponds to the probability.

Other features and advantages of the invention will be apparent from the detailed description, and from the claims.

### DETAILED DESCRIPTION

The present disclosure is based, in part, on the discovery that certain proteins and peptides in biological samples obtained from a pregnant female are differentially expressed in pregnant females that have an increased risk of preterm birth relative to matched controls. The present disclosure is further based, in part, on the unexepected discovery that panels combining one or more of these proteins and peptides can be utilized in methods of determining the probability for preterm birth in a pregnant female with relatively high sensitivity and specificity. These proteins and peptides dislosed herein serve as biomarkers for classifying test samples, predicting a probability of preterm birth, and/or monitoring of progress of preventative therapy in a pregnant female, either individually or in a panel of biomarkers.

The disclosure provides biomarker panels, methods and kits for determining the probability for preterm birth in a pregnant female. One major advantage of the present disclosure is that risk of developing preterm birth can be assessed early during pregnancy so that appropriate monitoring and clinical management to prevent preterm delivery can be initiated in a timely fashion. The present invention is of particular benefit to females lacking any risk factors for preterm birth and who would not otherwise be identified and treated.

By way of example, the present disclosure includes methods for generating a result useful in determining probability for preterm birth in a pregnant female by obtaining a dataset associated with a sample, where the dataset at least includes quantitative data about biomarkers and panels of biomarkers that have been identified as predictive of preterm birth, and inputting the dataset into an analytic process that uses the dataset to generate a result useful in determining probability for preterm birth in a pregnant female. As described further below, this quantitative data can include amino acids, peptides, polypeptides, proteins, nucleotides, nucleic acids, nucleosides, sugars, fatty acids, steroids, metabolites, carbohydrates, lipids, hormones, antibodies, regions of interest that serve as surrogates for biological macromolecules and combinations thereof.

In addition to the specific biomarkers identified in this disclosure, for example, by accession number in a public database, sequence, or reference, the invention also contemplates contemplates use of biomarker variants that are at least 90% or at least 95% or at least 97% identical to the exemplified sequences and that are now known or later discover and that have utility for the methods of the invention. These variants may represent polymorphisms, splice variants, mutations, and the like. In this regard, the instant specification discloses multiple art-known proteins in the context of the invention and provides exemplary accession numbers associated with one or more public databases as well as exemplary references to published journal articles relating to these art-known proteins. However, those skilled in the art appreciate that additional accession numbers and journal articles can easily be identified that can provide additional characteristics of the disclosed biomarkers and that the exemplified references are in no way limiting with regard to the disclosed biomarkers. As described herein, various techniques and reagents find use in the methods of the present invention. Suitable samples in the context of the present invention include, for example, blood, plasma, serum, amniotic fluid, vaginal excretions, saliva, and urine. In some embodiments, the biological sample is selected from the group consisting of whole blood, plasma, and serum. In a particular embodiment, the biological sample is serum. As described herein, biomarkers can be detected through a variety of assays and techniques known in the art. As further described herein, such assays include, without limitation, mass spectrometry (MS)-based assays, antibody-based assays as well as assays that combine aspects of the two.

Protein biomarkers associated with the probability for preterm birth in a pregnant female include, but are not limited to, one or more of the isolated biomarkers listed in Tables 1, 2, 3, 4, 6 and 7. In addition to the specific biomarkers, the disclosure further includes biomarker variants that are about 90%, about 95%, or about 97% identical to the exemplified sequences. Variants, as used herein, include polymorphisms, splice variants, mutations, and the like.

Additional markers can be selected from one or more risk indicia, including but not limited to, maternal characteristics, medical history, past pregnancy history, and obstetrical history. Such additional markers can include, for example, previous low birth weight or preterm delivery, multiple 2nd trimester spontaneous abortions, prior first trimester induced abortion, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low prepregnancy weight/low body mass index, diabetes, hypertension, urogenital infections. Demographic risk indicia for preterm birth can include, for example, race/ethnicity, single marital status, low socioeconomic status, maternal age, employment-related physical activity, occupational exposures and environment exposures. Further risk indicia can include, inadequate prenatal care, cigarette smoking, use of marijuana and other illicit drugs, cocaine use, alcohol consumption, caffeine intake, maternal weight gain, dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman R E, Butler A S, editors. Washington (DC): National Academies Press (US); 2007). Additional risk indicia useful for as markers can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

Provided herein are panels of isolated biomarkers comprising N of the biomarkers selected from the group listed in Tables 1, 2, 3, 4, 6 and 7. In the disclosed panels of biomarkers N can be a number selected from the group consisting of 2 to 24. In the disclosed methods, the number of biomarkers that are detected and whose levels are determined, can be 1, or more than 1, such as 2, 3, 4, 5, 6, 7, 8, 9, 10, 11, 12, 12, 14, 15, 16, 17, 18, 19, 20, 21, 22, 23, 24, 25 or more. In certain embodiments, the number of biomarkers that are detected, and whose levels are determined, can be 1, or more than 1, such as 2, 3, 4, 5, 6, 7, 8, 9, 10, or more. The methods of this disclosure are useful for determining the probability for preterm birth in a pregnant female.

While certain of the biomarkers listed in Tables 1, 2, 3, 4, 6 and 7 are useful alone for determining the probability for preterm birth in a pregnant female, methods are also described herein for the grouping of multiple subsets of the biomarkers that are each useful as a panel of three or more biomarkers. In some embodiments, the invention provides panels comprising N biomarkers, wherein N is at least three biomarkers. In other embodiments, N is selected to be any number from 3-23 biomarkers.

In yet other embodiments, N is selected to be any number from 2-5, 2-10, 2-15, 2-20, or 2-23. In other embodiments, N is selected to be any number from 3-5, 3-10, 3-15, 3-20, or 3-23. In other embodiments, N is selected to be any number from 4-5, 4-10, 4-15, 4-20, or 4-23. In other embodiments, N is selected to be any number from 5-10, 5-15, 5-20, or 5-23. In other embodiments, N is selected to be any number from 6-10, 6-15, 6-20, or 6-23. In other embodiments, N is selected to be any number from 7-10, 7-15, 7-20, or 7-23. In other embodiments, N is selected to be any number from 8-10, 8-15, 8-20, or 8-23. In other embodiments, N is selected to be any number from 9-10, 9-15, 9-20, or 9-23. In other embodiments, N is selected to be any number from 10-15, 10-20, or 10-23. It will be appreciated that N can be selected to encompass similar, but higher order, ranges.

In certain embodiments, the panel of isolated biomarkers comprises one or more, two or more, three or more, four or more, or five isolated biomarkers comprising an amino acid sequence selected from AFTECCVVASQLR, ELLESYIDGR, ITLPDFTGDLR, TDAPDLPEENQAR and SFRPFVPR.

In some embodiments, the panel of isolated biomarkers comprises one or more, two or more, or three of the isolated biomarkers consisting of an amino acid sequence selected from AFTECCVVASQLR, ELLESYIDGR, and ITLPDFTGDLR.

In some embodiments, the panel of isolated biomarkers comprises one or more peptides comprising a fragment from lipopolysaccharide-binding protein (LBP), Schumann et al., Science 249 (4975), 1429-1431 (1990) (UniProtKB/Swiss-Prot: P18428.3); prothrombin (THRB), Walz et al., Proc. Natl. Acad. Sci. U.S.A. 74 (5), 1969-1972 (1977) (NCBI Reference Sequence: NP_{—}000497.1); complement component C5 (C5 or CO5) Haviland, J. Immunol. 146 (1), 362-368 (1991) (GenBank: AAA51925.1); plasminogen (PLMN) Petersen et al., J. Biol. Chem. 265 (11), 6104-6111 (1990) (NCBI Reference Sequences: NP_{—}000292.1 NP_{—}001161810.1); and complement component C8 gamma chain (C8G or CO8G), Haefliger et al., Mol. Immunol. 28 (1-2), 123-131 (1991) (NCBI Reference Sequence: NP_{—}000597.2).

In some embodiments, the panel of isolated biomarkers comprises one or more peptides comprising a fragment from cell adhesion molecule with homology to complement component 1, q subcomponent, B chain (C1QB), Reid, Biochem. J. 179 (2), 367-371 (1979) (NCBI Reference Sequence: NP_{—}000482.3); fibrinogen beta chain (FIBB or FIB); Watt et al., Biochemistry 18 (1), 68-76 (1979) (NCBI Reference Sequences: NP_{—}001171670.1 and NP_{—}005132.2); C-reactive protein (CRP), Oliveira et al., J. Biol. Chem. 254 (2), 489-502 (1979) (NCBI Reference Sequence: NP_{—}000558.2); inter-alpha-trypsin inhibitor heavy chain H4 (ITIH4) Kim et al., Mol. Biosyst. 7 (5), 1430-1440 (2011) (NCBI Reference Sequences: NP_{—}001159921.1 and NP_{—}002209.2); chorionic somatomammotropin hormone (CSH) Selby et al., J. Biol. Chem. 259 (21), 13131-13138 (1984) (NCBI Reference Sequence: NP_{—}001308.1); and angiotensinogen (ANG or ANGT) Underwood et al., Metabolism 60(8):1150-7 (2011) (NCBI Reference Sequence: NP_{—}000020.1).

In additional embodiments, the invention provides a panel of isolated biomarkers comprising N of the biomarkers listed in Tables 1, 2, 3, 4, 6 and 7. In some embodiments, N is a number selected from the group consisting of 2 to 24. In additional embodiments, the biomarker panel comprises at least two of the isolated biomarkers selected from the group consisting of AFTECCVVASQLR, ELLESYIDGR, and ITLPDFTGDLR. In additional embodiments, the biomarker panel comprises at least two of the isolated biomarkers selected from the group consisting of AFTECCVVASQLR, ELLESYIDGR, ITLPDFTGDLR, TDAPDLPEENQAR and SFRPFVPR.

In further embodiments, the biomarker panel comprises at least two of the isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G). In another embodiment, the invention provides a biomarker panel comprising at least three isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G).

In some embodiments, the invention provides a biomarker panel comprising lipopolysaccharide-binding protein (LBP), prothrombin (THRB), complement component C5 (C5 or CO5), plasminogen (PLMN), complement component C8 gamma chain (C8G or CO8G), complement component 1, q subcomponent, B chain (C1QB), fibrinogen beta chain (FIBB or FIB), C-reactive protein (CRP), inter-alpha-trypsin inhibitor heavy chain H4 (ITIH4), chorionic somatomammotropin hormone (CSH), and angiotensinogen (ANG or ANGT). In another aspect, the invention provides a biomarker panel comprising at least two isolated biomarkers selected from the group consisting of lipopolysaccharide-binding protein (LBP), prothrombin (THRB), complement component C5 (C5 or CO5), plasminogen (PLMN), complement component C8 gamma chain (C8G or CO8G), complement component 1, q subcomponent, B chain (C1QB), fibrinogen beta chain (FIBB or FIB), C-reactive protein (CRP), inter-alpha-trypsin inhibitor heavy chain H4 (ITIH4), chorionic somatomammotropin hormone (CSH), and angiotensinogen (ANG or ANGT).

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about,” particularly in reference to a given quantity, is meant to encompass deviations of plus or minus five percent.

As used in this application, including the appended claims, the singular forms “a,” “an,” and “the” include plural references, unless the content clearly dictates otherwise, and are used interchangeably with “at least one” and “one or more.”

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

As used herein, the term “panel” refers to a composition, such as an array or a collection, comprising one or more biomarkers. The term can also refer to a profile or index of expression patterns of one or more biomarkers described herein. The number of biomarkers useful for a biomarker panel is based on the sensitivity and specificity value for the particular combination of biomarker values.

As used herein, and unless otherwise specified, the terms “isolated” and “purified” generally describes a composition of matter that has been removed from its native environment (e.g., the natural environment if it is naturally occurring), and thus is altered by the hand of man from its natural state. An isolated protein or nucleic acid is distinct from the way it exists in nature.

The term “biomarker” refers to a biological molecule, or a fragment of a biological molecule, the change and/or the detection of which can be correlated with a particular physical condition or state. The terms “marker” and “biomarker” are used interchangeably throughout the disclosure. For example, the biomarkers of the present invention are correlated with an increased likelihood of preterm birth. Such biomarkers include, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins). The term also encompasses portions or fragments of a biological molecule, for example, peptide fragment of a protein or polypeptide that comprises at least 5 consecutive amino acid residues, at least 6 consecutive amino acid residues, at least 7 consecutive amino acid residues, at least 8 consecutive amino acid residues, at least 9 consecutive amino acid residues, at least 10 consecutive amino acid residues, at least 11 consecutive amino acid residues, at least 12 consecutive amino acid residues, at least 13 consecutive amino acid residues, at least 14 consecutive amino acid residues, at least 15 consecutive amino acid residues, at least 5 consecutive amino acid residues, at least 16 consecutive amino acid residues, at least 17 consecutive amino acid residues, at least 18 consecutive amino acid residues, at least 19 consecutive amino acid residues, at least 20 consecutive amino acid residues, at least 21 consecutive amino acid residues, at least 22 consecutive amino acid residues, at least 23 consecutive amino acid residues, at least 24 consecutive amino acid residues, at least 25 consecutive amino acid residues, or more consecutive amino acid residues.

The invention also provides a method of determining probability for preterm birth in a pregnant female, the method comprising detecting a measurable feature of each of N biomarkers selected from the biomarkers listed in Tables 1, 2, 3, 4, 6 and 7 in a biological sample obtained from the pregnant female, and analyzing the measurable feature to determine the probability for preterm birth in the pregnant female. As disclosed herein, a measurable feature comprises fragments or derivatives of each of said N biomarkers selected from the biomarkers listed in Tables 1, 2, 3, 4, 6 and 7. In some embodiments of the disclosed methods detecting a measurable feature comprises quantifying an amount of each of N biomarkers selected from the biomarkers listed in Tables 1, 2, 3, 4, 6 and 7, combinations or portions and/or derivatives thereof in a biological sample obtained from said pregnant female.

In some embodiments, the method of determining probability for preterm birth in a pregnant female comprises detecting a measurable feature of each of N biomarkers, wherein N is selected from the group consisting of 2 to 24. In further embodiments, the disclosed methods of determining probability for preterm birth in a pregnant female comprises detecting a measurable feature of each of at least two isolated biomarkers selected from the group consisting of AFTECCVVASQLR, ELLESYIDGR, and ITLPDFTGDLR.

In additional embodiments, the method of determining probability for preterm birth in a pregnant female comprises detecting a measurable feature of each of at least two isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G).

In further embodiments, the disclosed method of determining probability for preterm birth in a pregnant female comprises detecting a measurable feature of each of at least two isolated biomarkers selected from the group consisting of lipopolysaccharide-binding protein (LBP), prothrombin (THRB), complement component C5 (C5 or CO5), plasminogen (PLMN), complement component C8 gamma chain (C8G or CO8G), complement component 1, q subcomponent, B chain (C1QB), fibrinogen beta chain (FIBB or FIB), C-reactive protein (CRP), inter-alpha-trypsin inhibitor heavy chain H4 (ITIH4), chorionic somatomammotropin hormone (CSH), and angiotensinogen (ANG or ANGT).

In additional embodiments, the methods of determining probability for preterm birth in a pregnant female further encompass detecting a measurable feature for one or more risk indicia associated with preterm birth. In additional embodiments the risk indicia are selected form the group consisting of previous low birth weight or preterm delivery, multiple 2nd trimester spontaneous abortions, prior first trimester induced abortion, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low prepregnancy weight/low body mass index, diabetes, hypertension, and urogenital infections.

A “measurable feature” is any property, characteristic or aspect that can be determined and correlated with the probability for preterm birth in a subject. For a biomarker, such a measurable feature can include, for example, the presence, absence, or concentration of the biomarker, or a fragment thereof, in the biological sample, an altered structure, such as, for example, the presence or amount of a post-translational modification, such as oxidation at one or more positions on the amino acid sequence of the biomarker or, for example, the presence of an altered conformation in comparison to the conformation of the biomarker in normal control subjects, and/or the presence, amount, or altered structure of the biomarker as a part of a profile of more than one biomarker. In addition to biomarkers, measurable features can further include risk indicia including, for example, maternal characteristics, medical history, past pregnancy history, obstetrical history. For a risk indicium, a measurable feature can include, for example, previous low birth weight or preterm delivery, multiple 2nd trimester spontaneous abortions, prior first trimester induced abortion, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low prepregnancy weight/low body mass index, diabetes, hypertension, urogenital infections.

In some embodiments of the disclosed methods of determining probability for preterm birth in a pregnant female, the probability for preterm birth in the pregnant female is calculated based on the quantified amount of each of N biomarkers selected from the biomarkers listed in Tables 1, 2, 3, 4, 6 and 7. In some embodiments, the disclosed methods for determining the probability of preterm birth encompass detecting and/or quantifying one or more biomarkers using mass sprectrometry, a capture agent or a combination thereof.

In some embodiments, the disclosed methods of determining probability for preterm birth in a pregnant female encompass an initial step of providing a biomarker panel comprising N of the biomarkers listed in Tables 1, 2, 3, 4, 6 and 7. In additional embodiments, the disclosed methods of determining probability for preterm birth in a pregnant female encompass an initial step of providing a biological sample from the pregnant female.

In some embodiments, the disclosed methods of determining probability for preterm birth in a pregnant female encompass communicating the probability to a health care provider. In additional embodiments, the communication informs a subsequent treatment decision for the pregnant female. In some embodiments, the method of determining probability for preterm birth in a pregnant female encompasses the additional feature of expressing the probability as a risk score.

As used herein, the term “risk score” refers to a score that can be assigned based on comparing the amount of one or more biomarkers in a biological sample obtained from a pregnant female to a standard or reference score that represents an average amount of the one or more biomarkers calculated from biological samples obtained from a random pool of pregnant females. Because the level of a biomarker may not be static throughout pregnancy, a standard or reference score has to have been obtained for the gestational time point that corresponds to that of the pregnant female at the time the sample was taken. The standard or reference score can be predetermined and built into a predictor model such that the comparison is indirect rather than actually performed every time the probability is determined for a subject. A risk score can be a standard (e.g., a number) or a threshold (e.g., a line on a graph). The value of the risk score correlates to the deviation, upwards or downwards, from the average amount of the one or more biomarkers calculated from biological samples obtained from a random pool of pregnant females. In certain embodiments, if a risk score is greater than a standard or reference risk score, the pregnant female can have an increased likelihood of preterm birth. In some embodiments, the magnitude of a pregnant female's risk score, or the amount by which it exceeds a reference risk score, can be indicative of or correlated to that pregnant female's level of risk.

In the context of the present invention, the term “biological sample,” encompasses any sample that is taken from pregnant female and contains one or more of the biomarkers listed in Table 1. Suitable samples in the context of the present invention include, for example, blood, plasma, serum, amniotic fluid, vaginal excretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles. In a particular embodiment, the biological sample is serum.

Preterm birth refers to delivery or birth at a gestational age less than 37 completed weeks. Other commonly used subcategories of preterm birth have been established and delineate moderately preterm (birth at 33 to 36 weeks of gestation), very preterm (birth at <33 weeks of gestation), and extremely preterm (birth at ≦28 weeks of gestation). Gestational age is a proxy for the extent of fetal development and the fetus's readiness for birth. Gestational age has typically been defined as the length of time from the date of the last normal menses to the date of birth. However, obstetric measures and ultrasound estimates also can aid in determining gestational age. Preterm births have generally been classified into two separate subgroups. One, spontaneous preterm births are those occurring subsequent to spontaneous onset of preterm labor or preterm premature rupture of membranes regardless of subsequent labor augmentation or cesarean delivery. Two, indicated preterm births are those occurring following induction or cesarean section for one or more conditions that the woman's caregiver determines to threaten the health or life of the mother and/or fetus. In some embodiments, the methods disclosed herein are directed to determining the probability for spontaneous preterm birth.

In some embodiments, the pregnant female was between 17 and 28 weeks of gestation at the time the biological sample was collected. In other embodiments, the pregnant female was between 16 and 29 weeks, between 17 and 28 weeks, between 18 and 27 weeks, between 19 and 26 weeks, between 20 and 25 weeks, between 21 and 24 weeks, or between 22 and 23 weeks of gestation at the time the biological sample was collected. In further embodiments, the pregnant female was between about 17 and 22 weeks, between about 16 and 22 weeks between about 22 and 25 weeks, between about 13 and 25 weeks, between about 26 and 28, or between about 26 and 29 weeks of gestation at the time the biological sample was collected. Accordingly, the gestational age of a pregnant female at the time the biological sample is collected can be 15, 16, 17, 18, 19, 20, 21, 22, 23, 24, 25, 26, 27, 28, 29 or 30 weeks.

In some embodiments of the claimed methods the measurable feature comprises fragments or derivatives of each of the N biomarkers selected from the biomarkers listed in Table 1. In additional embodiments of the claimed methods, detecting a measurable feature comprises quantifying an amount of each of N biomarkers selected from the biomarkers listed in Table 1, combinations or portions and/or derivatives thereof in a biological sample obtained from said pregnant female.

The term “amount” or “level” as used herein refers to a quantity of a biomarker that is detectable or measurable in a biological sample and/or control. The quantity of a biomarker can be, for example, a quantity of polypeptide, the quantity of nucleic acid, or the quantity of a fragment or surrogate. The term can alternatively include combinations thereof. The term “amount” or “level” of a biomarker is a measurable feature of that biomarker.

In some embodiments, calculating the probability for preterm birth in a pregnant female is based on the quantified amount of each of N biomarkers selected from the biomarkers listed in Table 1. Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of biomarkers, peptides, polypeptides, proteins and/or fragments thereof and optionally of the one or more other biomarkers or fragments thereof in samples. In some embodiments, detection and/or quantification of one or more biomarkers comprises an assay that utilizes a capture agent. In further ambodiments, the capture agent is an antibody, antibody fragment, nucleic acid-based protein binding reagent, small molecule or variant thereof. In additional embodimentns, the assay is an enzyme immunoassay (EIA), enzyme-linked immunosorbent assay (ELISA), and radioimmunoassay (RIA). In some embodiments, detection and/or quantification of one or more biomarkers further comprises mass spectrometry (MS). In yet further embodiments, the mass spectrometry is co-immunoprecitipation-mass spectrometry (co-IP MS), where coimmunoprecipitation, a technique suitable for the isolation of whole protein complexes is followed by mass spectrometric analysis.

As used herein, the term “mass spectrometer” refers to a device able to volatilize/ionize analytes to form gas-phase ions and determine their absolute or relative molecular masses. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods disclosed herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000; Biemann 1990. Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more biomarkers. Such quantitiative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS). Other methods useful in this context include isotope-coded affinity tag (ICAT) followed by chromatography and MS/MS.

As used herein, the terms “multiple reaction monitoring (MRM)” or “selected reaction monitoring (SRM)” refer to an MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment, A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, included in the assay can be standards that correspond to the analytes of interest (e.g., same amino acid sequence), but differ by the inclusion of stable isotopes. Stable isotopic standards (SIS) can be incorporated into the assay at precise levels and used to quantify the corresponding unknown analyte. An additional level of specificity is contributed by the co-elution of the unknown analyte and its corresponding SIS and properties of their transitions (e.g., the similarity in the ratio of the level of two transitions of the unknown and the ratio of the two transitions of its corresponding SIS).

Mass spectrometry assays, instruments and systems suitable for biomarker peptide analysis can include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ₙ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APC-MS)ₙ; atmospheric pressure photoionization mass spectrometry (APPI-MS); APPI-MS/MS; and APPI-(MS)ₙ. Peptide ion fragmentation in tandem MS (MS/MS) arrangements can be achieved using manners established in the art, such as, e.g., collision induced dissociation (CID). As described herein, detection and quantification of biomarkers by mass spectrometry can involve multiple reaction monitoring (MRM), such as described among others by Kuhn et al. Proteomics 4: 1175-86 (2004). Scheduled multiple-reaction-monitoring (Scheduled MRM) mode acquisition during LC-MS/MS analysis enhances the sensitivity and accuracy of peptide quantitation. Anderson and Hunter, Molecular and Cellular Proteomics 5(4):573 (2006). As described herein, mass spectrometry-based assays can be advantageously combined with upstream peptide or protein separation or fractionation methods, such as for example with the chromatographic and other methods described herein below.

A person skilled in the art will appreciate that a number of methods can be used to determine the amount of a biomarker, including mass spectrometry approaches, such as MS/MS, LC-MS/MS, multiple reaction monitoring (MRM) or SRM and product-ion monitoring (PIM) and also including antibody based methods such as immunoassays such as Western blots, enzyme-linked immunosorbant assay (ELISA), immunopercipitation, immunohistochemistry, immunofluorescence, radioimmunoassay, dot blotting, and FACS. Accordingly, in some embodiments, determining the level of the at least one biomarker comprises using an immunoassay and/or mass spectrometric methods. In additional embodiments, the mass spectrometric methods are selected from MS, MS/MS, LC-MS/MS, SRM, PIM, and other such methods that are known in the art. In other embodiments, LC-MS/MS further comprises 1D LC-MS/MS, 2D LC-MS/MS or 3D LC-MS/MS. Immunoassay techniques and protocols are generally known to those skilled in the art (Price and Newman, Principles and Practice of Immunoassay, 2nd Edition, Grove's Dictionaries, 1997; and Gosling, Immunoassays: A Practical Approach, Oxford University Press, 2000.) A variety of immunoassay techniques, including competitive and non-competitive immunoassays, can be used (Self et al., Curr. Opin. Biotechnol., 7:60-65 (1996).

In further embodiments, the immunoassay is selected from Western blot, ELISA, immunopercipitation, immunohistochemistry, immunofluorescence, radioimmunoassay (RIA), dot blotting, and FACS. In certain embodiments, the immunoassay is an ELISA. In yet a further embodiment, the ELISA is direct ELISA (enzyme-linked immunosorbent assay), indirect ELISA, sandwich ELISA, competitive ELISA, multiplex ELISA, ELISPOT technologies, and other similar techniques known in the art. Principles of these immunoassay methods are known in the art, for example John R. Crowther, The ELISA Guidebook, 1st ed., Humana Press 2000, ISBN 0896037282. Typically ELISAs are performed with antibodies but they can be performed with any capture agents that bind specifically to one or more biomarkers of the invention and that can be detected. Multiplex ELISA allows simultaneous detection of two or more analytes within a single compartment (e.g., microplate well) usually at a plurality of array addresses (Nielsen and Geierstanger 2004. J Immunol Methods 290: 107-20 (2004) and Ling et al. 2007. Expert Rev Mol Diagn 7: 87-98 (2007)).

In some embodiments, Radioimmunoassay (RIA) can be used to detect one or more biomarkers in the methods of the invention. Radioimmunoassay) is a competition-based assay that is erll known in the art and involves mixing known quantities of radioactavely-labelled (e.g., ¹²⁵I or ¹³¹I-labelled) target analyte with antibody specific for the analyte, then adding non-labelled analyte from a sample and measuring the amount of labelled analyte that is displaced (see, e.g., An Introduction to Radioimmunoassay and Related Techniques, by Chard T, ed., Elsevier Science 1995, ISBN 0444821198 for guidance).

A detectable label can be used in the assays described herein for direct or indirect detection of the biomarkers in the methods of the invention. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label based on the assay detection of the biomarkers in the methods of the invention. Suitable detectable labels include, but are not limited to, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

For mass-sectrometry based analysis, differential tagging with isotopic reagents, e.g., isotope-coded affinity tags (ICAT) or the more recent variation that uses isobaric tagging reagents, iTRAQ (Applied Biosystems, Foster City, Calif.), followed by multidimensional liquid chromatography (LC) and tandem mass spectrometry (MS/MS) analysis can provide a further methodology in practicing the methods of the inventon.

A chemiluminescence assay using a chemiluminescent antibody can be used for sensitive, non-radioactive detection of protein levels. An antibody labeled with fluorochrome also can be suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), beta-galactosidase, urease, and the like. Detection systems using suitable substrates for horseradish-peroxidase, alkaline phosphatase, beta.-galactosidase are well known in the art.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, assays used to practice the invention can be automated or performed robotically, and the signal from multiple samples can be detected simultaneously.

In some embodiments, the methods described herein encompass quantification of the biomarkers using mass spectrometry (MS). In further embodiments, the mass spectrometry can be liquid chromatography-mass spectrometry (LC-MS), multiple reaction monitoring (MRM) or selected reaction monitoring (SRM). In additional embodiments, the MRM or SRM can further encompass scheduled MRM or scheduled SRM.

As described above, chromatography can also be used in practicing the methods of the invention. Chromatography encompasses methods for separating chemical substances and generally involves a process in which a mixture of analytes is carried by a moving stream of liquid or gas (“mobile phase”) and separated into components as a result of differential distribution of the analytes as they flow around or over a stationary liquid or solid phase (“stationary phase”), between the mobile phase and said stationary phase. The stationary phase can be usually a finely divided solid, a sheet of filter material, or a thin film of a liquid on the surface of a solid, or the like. Chromatography is well understood by those skilled in the art as a technique applicable for the separation of chemical compounds of biological origin, such as, e.g., amino acids, proteins, fragments of proteins or peptides, etc.

Chromatography can be columnar (i.e., wherein the stationary phase is deposited or packed in a column), preferably liquid chromatography, and yet more preferably high-performance liquid chromatography (HPLC). Particulars of chromatography are well known in the art (Bidlingmeyer, Practical HPLC Methodology and Applications, John Wiley & Sons Inc., 1993). Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like. Chromatography, including single-, two- or more-dimensional chromatography, can be used as a peptide fractionation method in conjunction with a further peptide analysis method, such as for example, with a downstream mass spectrometry analysis as described elsewhere in this specification.

Further peptide or polypeptide separation, identification or quantification methods can be used, optionally in conjunction with any of the above described analysis methods, for measuring biomarkers in the present disclosure. Such methods include, without limitation, chemical extraction partitioning, isoelectric focusing (IEF) including capillary isoelectric focusing (LIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In the context of the invention, the term “capture agent” refers to a compound that can specifically bind to a target, in particular a biomarker. The term includes antibodies, antibody fragments, nucleic acid-based protein binding reagents (e.g. aptamers, Slow Off-rate Modified Aptamers (SOMAmer™)), protein-capture agents, natural ligands (i.e. a hormone for its receptor or vice versa), small molecules or variants thereof.

Capture agents can be configured to specifically bind to a target, in particular a biomarker. Capture agents can include but are not limited to organic molecules, such as polypeptides, polynucleotides and other non polymeric molecules that are identifiable to a skilled person. In the embodiments disclosed herein, capture agents include any agent that can be used to detect, purify, isolate, or enrich a target, in particular a biomarker. Any art-known affinity capture technologies can be used to selectively isolate and enrich/concentrate biomarkers that are components of complex mixtures of biological media for use in the disclosed methods.

Antibody capture agents that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986). Antibody capture agents can be any immunoglobulin or derivative thereof, whether natural or wholly or partially synthetically produced. All derivatives thereof which maintain specific binding ability are also included in the term. Antibody capture agents have a binding domain that is homologous or largely homologous to an immunoglobulin binding domain and can be derived from natural sources, or partly or wholly synthetically produced. Antibody capture agents can be monoclonal or polyclonal antibodies. In some embodiments, an antibody is a single chain antibody. Those of ordinary skill in the art will appreciate that antibodies can be provided in any of a variety of forms including, for example, humanized, partially humanized, chimeric, chimeric humanized, etc. Antibody capture agents can be antibody fragments including, but not limited to, Fab, Fab′, F(ab′)2, scFv, Fv, dsFv diabody, and Fd fragments. An antibody capture agent can be produced by any means. For example, an antibody capture agent can be enzymatically or chemically produced by fragmentation of an intact antibody and/or it can be recombinantly produced from a gene encoding the partial antibody sequence. An antibody capture agent can comprise a single chain antibody fragment. Alternatively or additionally, antibody capture agent can comprise multiple chains which are linked together, for example, by disulfide linkages; and, any functional fragments obtained from such molecules, wherein such fragments retain specific-binding properties of the parent antibody molecule. Because of their smaller size as functional components of the whole molecule, antibody fragments can offer advantages over intact antibodies for use in certain immunochemical techniques and experimental applications.

Suitable capture agents useful for practicing the invention also include aptamers. Aptamers are oligonucleotide sequences that can bind to their targets specifically via unique three dimensional (3-D) structures. An aptamer can include any suitable number of nucleotides and different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Use of an aptamer capture agent can include the use of two or more aptamers that specifically bind the same biomarker. An aptamer can include a tag. An aptamer can be identified using any known method, including the SELEX (systematic evolution of ligands by exponential enrichment), process. Once identified, an aptamer can be prepared or synthesized in accordance with any known method, including chemical synthetic methods and enzymatic synthetic methods and used in a variety of applications for biomarker detection. Liu et al., Curr Med. Chem. 18(27):4117-25 (2011). Capture agents useful in practicing the methods of the invention also include SOMAmers (Slow Off-Rate Modified Aptamers) known in the art to have improved off-rate characteristics. Brody et al., J Mol. Biol. 422(5):595-606 (2012). SOMAmers can be generated using any known method, including the SELEX method.

It is understood by those skilled in the art that biomarkers can be modified prior to analysis to improve their resolution or to determine their identity. For example, the biomarkers can be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

It is further appreciated in the art that biomarkers in a sample can be captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules can be antibodies, peptides, peptoids, aptamers, small molecule ligands or other protein-binding capture agents attached to the surface of particles. Each protein-binding molecule can include unique detectable label that is coded such that it can be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes.

In another aspect, biochips can be used for capture and detection of the biomarkers of the invention. Many protein biochips are known in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture agent bound there. The capture agent can be a biological molecule, such as a polypeptide or a nucleic acid, which captures other biomarkers in a specific manner. Alternatively, the capture agent can be a chromatographic material, such as an anion exchange material or a hydrophilic material. Examples of protein biochips are well known in the art.

Measuring mRNA in a biological sample can be used as a surrogate for detection of the level of the corresponding protein biomarker in a biological sample. Thus, any of the biomarkers or biomarker panels described herein can also be detected by detecting the appropriate RNA. Levels of mRNA can measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

Some embodiments disclosed herein relate to diagnostic and prognostic methods of determining the probability for preterm birth in a pregnant female. The detection of the level of expression of one or more biomarkers and/or the determination of a ratio of biomarkers can be used to determine the probability for preterm birth in a pregnant female. Such detection methods can be used, for example, for early diagnosis of the condition, to determine whether a subject is predisposed to preterm birth, to monitor the progress of preterm birth or the progress of treatment protocols, to assess the severity of preterm birth, to forecast the outcome of preterm birth and/or prospects of recovery or birth at full term, or to aid in the determination of a suitable treatment for preterm birth.

The quantitation of biomarkers in a biological sample can be determined, without limitation, by the methods described above as well as any other method known in the art. The quantitative data thus obtained is then subjected to an analytic classification process. In such a process, the raw data is manipulated according to an algorithm, where the algorithm has been pre-defined by a training set of data, for example as described in the examples provided herein. An algorithm can utilize the training set of data provided herein, or can utilize the guidelines provided herein to generate an algorithm with a different set of data.

In some embodiments, analyzing a measurable feature to determine the probability for preterm birth in a pregnant female encompasses the use of a predictive model. In further embodiments, analyzing a measurable feature to determine the probability for preterm birth in a pregnant female encompasses comparing said measurable feature with a reference feature. As those skilled in the art can appreciate, such comparison can be a direct comparison to the reference feature or an indirect comparison where the reference feature has been incorporated into the predictive model. In further embodiments, analyzing a measurable feature to determine the probability for preterm birth in a pregnant female encompasses one or more of a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, or a combination thereof. In particular embodiments, the analysis comprises logistic regression.

An analytic classification process can use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc.

Classification can be made according to predictive modeling methods that set a threshold for determining the probability that a sample belongs to a given class. The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, or higher.

The raw data can be initially analyzed by measuring the values for each biomarker, usually in triplicate or in multiple triplicates. The data can be manipulated, for example, raw data can be transformed using standard curves, and the average of triplicate measurements used to calculate the average and standard deviation for each patient. These values can be transformed before being used in the models, e.g. log-transformed, Box-Cox transformed (Box and Cox, Royal Stat. Soc., Series B, 26:211-246 (1964). The data are then input into a predictive model, which will classify the sample according to the state. The resulting information can be communicated to a patient or health care provider.

To generate a predictive model for preterm birth, a robust data set, comprising known control samples and samples corresponding to the preterm birth classification of interest is used in a training set. A sample size can be selected using generally accepted criteria. As discussed above, different statistical methods can be used to obtain a highly accurate predictive model. Examples of such analysis are provided in Example 2.

In one embodiment, hierarchical clustering is performed in the derivation of a predictive model, where the Pearson correlation is employed as the clustering metric. One approach is to consider a preterm birth dataset as a “learning sample” in a problem of “supervised learning.” CART is a standard in applications to medicine (Singer, Recursive Partitioning in the Health Sciences, Springer (1999)) and can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach led to what is termed FlexTree (Huang, Proc. Nat. Acad. Sci. U.S.A 101:10529-10534 (2004)). FlexTree performs very well in simulations and when applied to multiple forms of data and is useful for practicing the claimed methods. Software automating FlexTree has been developed. Alternatively, LARTree or LART can be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451 (2004). See, also, Huang et al., Proc. Natl. Acad. Sci. USA. 101(29):10529-34 (2004). Other methods of analysis that can be used include logic regression. One method of logic regression Ruczinski, Journal of Computational and Graphical Statistics 12:475-512 (2003). Logic regression resembles CART in that its classifier can be displayed as a binary tree. It is different in that each node has Boolean statements about features that are more general than the simple “and” statements produced by CART.

Another approach is that of nearest shrunken centroids (Tibshirani, Proc. Natl. Acad. Sci. U.S.A 99:6567-72 (2002)). The technology is k-means-like, but has the advantage that by shrinking cluster centers, one automatically selects features, as is the case in the lasso, to focus attention on small numbers of those that are informative. The approach is available as PAM software and is widely used. Two further sets of algorithms that can be used are random forests (Breiman, Machine Learning 45:5-32 (2001)) and MART (Hastie, The Elements of Statistical Learning, Springer (2001)). These two methods are known in the art as “committee methods,” that involve predictors that “vote” on outcome.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (Tusher et al., Proc. Natl. Acad. Sci. U.S.A 98, 5116-21 (2001)). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles. Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pair wise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors in a time-to-event analysis (survival analysis), where the event is the occurrence of preterm birth, and subjects with no event are considered censored at the time of giving birth. Given the specific pregnancy outcome (preterm birth event or no event), the random lengths of time each patient will be observed, and selection of proteomic and other features, a parametric approach to analyzing survival can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and corresponding functions are available with this model.

In addition the Cox models can be used, especially since reductions of numbers of covariates to manageable size with the lasso will significantly simplify the analysis, allowing the possibility of a nonparametric or semi-parametric approach to prediction of time to preterm birth. These statistical tools are known in the art and applicable to all manner of proteomic data. A set of biomarker, clinical and genetic data that can be easily determined, and that is highly informative regarding the probability for preterm birth and predicted time to a preterm birth event in said pregnant female is provided. Also, algorithms provide information regarding the probability for preterm birth in the pregnant female.

In the development of a predictive model, it can be desirable to select a subset of markers, i.e. at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model. The selection of a number of informative markers for building classification models requires the definition of a performance metric and a user-defined threshold for producing a model with useful predictive ability based on this metric. For example, the performance metric can be the AUC, the sensitivity and/or specificity of the prediction as well as the overall accuracy of the prediction model.

As will be understood by those skilled in the art, an analytic classification process can use any one of a variety of statistical analytic methods to manipulate the quantitative data and provide for classification of the sample. Examples of useful methods include, without limitation, linear discriminant analysis, recursive feature elimination, a prediction analysis of microarray, a logistic regression, a CART algorithm, a FlexTree algorithm, a LART algorithm, a random forest algorithm, a MART algorithm, and machine learning algorithms.

As described in Example 2, various methods are used in a training model. The selection of a subset of markers can be for a forward selection or a backward selection of a marker subset. The number of markers can be selected that will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

TABLE 1
Transitions with p-values less than 0.05 in univariate Cox Proportional
Hazards analyses to predict Gestational Age at Birth

| | | p-value |
|---|---|---|
| | | Cox |
| Transition | Protein | univariate |
| ITLPDFTGDLR_624.34_920.4 | LBP_HUMAN | 0.006 |
| ELLESYIDGR_597.8_710.3 | THRB_HUMAN | 0.006 |
| TDAPDLPEENQAR_728.34_613.3 | CO5_HUMAN | 0.007 |
| AFTECCVVASQLR_770.87_574.3 | CO5_HUMAN | 0.009 |
| SFRPFVPR_335.86_272.2 | LBP_HUMAN | 0.011 |
| ITLPDFTGDLR_624.34_288.2 | LBP_HUMAN | 0.012 |
| SFRPFVPR_335.86_635.3 | LBP_HUMAN | 0.015 |
| ELLESYIDGR_597.8_839.4 | THRB_HUMAN | 0.018 |
| LEQGENVFLQATDK_796.4_822.4 | C1QB_HUMAN | 0.019 |
| ETAASLLQAGYK_626.33_679.4 | THRB_HUMAN | 0.021 |
| VTGWGNLK_437.74_617.3 | THRB_HUMAN | 0.021 |
| EAQLPVIENK_570.82_699.4 | PLMN_HUMAN | 0.023 |
| EAQLPVIENK_570.82_329.1 | PLMN_HUMAN | 0.023 |
| FLQEQGHR_338.84_497.3 | CO8G_HUMAN | 0.025 |
| IRPFFPQQ_516.79_661.4 | FIBB_HUMAN | 0.028 |
| ETAASLLQAGYK_626.33_879.5 | THRB_HUMAN | 0.029 |
| AFTECCVVASQLR_770.87_673.4 | CO5_HUMAN | 0.030 |
| TLLPVSKPEIR_418.26_288.2 | CO5_HUMAN | 0.030 |
| LSSPAVITDK_515.79_743.4 | PLMN_HUMAN | 0.033 |
| YEVQGEVFTKPQLWP_910.96_392.2 | CRP_HUMAN | 0.036 |
| LQGTLPVEAR_542.31_571.3 | CO5_HUMAN | 0.036 |
| VRPQQLVK_484.31_609.3 | ITIH4_HUMAN | 0.036 |
| IEEIAAK_387.22_531.3 | CO5_HUMAN | 0.041 |
| TLLPVSKPEIR_418.26_514.3 | CO5_HUMAN | 0.042 |
| VQEAHLTEDQIFYFPK_655.66_701.4 | CO8G_HUMAN | 0.047 |
| ISLLLIESWLEPVR_834.49_371.2 | CSH_HUMAN | 0.048 |
| ALQDQLVLVAAK_634.88_289.2 | ANGT_HUMAN | 0.048 |
| YEFLNGR_449.72_293.1 | PLMN_HUMAN | 0.049 |

TABLE 2
Transitions selected by the Cox stepwise AIC analysis

| Transition | coef | exp(coef) | se(coef) | z | Pr(>\|z\|) |
|---|---|---|---|---|---|
| Collection.Window.GA.in.Days | 1.28E−01 | 1.14E+00 | 2.44E−02 | 5.26 | 1.40E−07 |
| ITLPDFTGDLR_624.34_920.4 | 2.02E+00 | 7.52E+00 | 1.14E+00 | 1.77 | 0.07667 |
| TPSAAYLWVGTGASEAEK_919.45_849.4 | 2.85E+01 | 2.44E+12 | 3.06E+00 | 9.31 | <2e−16 |
| TATSEYQTFFNPR_781.37_386.2 | 5.14E+00 | 1.70E+02 | 6.26E−01 | 8.21 | 2.20E−16 |
| TASDFITK_441.73_781.4 | −1.25E+00 | 2.86E−01 | 1.58E+00 | −0.79 | 0.42856 |
| IITGLLEFEVYLEYLQNR_738.4_530.3 | 1.30E+01 | 4.49E+05 | 1.45E+00 | 9 | <2e−16 |
| IIGGSDADIK_494.77_762.4 | −6.43E+01 | 1.16E−28 | 6.64E+00 | −9.68 | <2e−16 |
| YTTEIIK_434.25_603.4 | 6.96E+01 | 1.75E+30 | 7.06E+00 | 9.86 | <2e−16 |
| EDTPNSVWEPAK_686.82_315.2 | 7.91E+00 | 2.73E+03 | 2.66E+00 | 2.98 | 0.00293 |
| LYYGDDEK_501.72_726.3 | 8.74E+00 | 6.23E+03 | 1.57E+00 | 5.57 | 2.50E−08 |
| VRPQQLVK_484.31_609.3 | 4.64E+01 | 1.36E+20 | 3.97E+00 | 11.66 | <2e−16 |
| GGEIEGFR_432.71_379.2 | −3.33E+00 | 3.57E−02 | 2.19E+00 | −1.52 | 0.12792 |
| DGSPDVTTADIGANTPDATK_973.45_844.4 | −1.52E+01 | 2.51E−07 | 1.41E+00 | −10.8 | <2e−16 |
| VQEAHLTEDQIFYFPK_655.66_391.2 | −2.02E+01 | 1.77E−09 | 2.45E+00 | −8.22 | 2.20E−16 |
| VEIDTK_352.7_476.3 | 7.06E+00 | 1.17E+03 | 1.45E+00 | 4.86 | 1.20E−06 |
| AVLTIDEK_444.76_605.3 | 7.85E+00 | 2.56E+03 | 9.46E−01 | 8.29 | <2e−16 |
| FSVVYAK_407.23_579.4 | −2.44E+01 | 2.42E−11 | 3.08E+00 | −7.93 | 2.20E−15 |
| YYLQGAK_421.72_516.3 | −1.82E+01 | 1.22E−08 | 2.45E+00 | −7.44 | 1.00E−13 |
| EENFYVDETTVVK_786.88_259.1 | −1.90E+01 | 5.36E−09 | 2.71E+00 | −7.03 | 2.00E−12 |
| YGFYTHVFR_397.2_421.3 | 1.90E+01 | 1.71E+08 | 2.73E+00 | 6.93 | 4.20E−12 |
| HTLNQIDEVK_598.82_951.5 | 1.03E+01 | 3.04E+04 | 2.11E+00 | 4.89 | 9.90E−07 |
| AFIQLWAFDAVK_704.89_836.4 | 1.08E+01 | 4.72E+04 | 2.59E+00 | 4.16 | 3.20E−05 |
| SGFSFGFK_438.72_585.3 | 1.35E+01 | 7.32E+05 | 2.56E+00 | 5.27 | 1.40E−07 |
| GWVTDGFSSLK_598.8_854.4 | −3.12E+00 | 4.42E−02 | 9.16E−01 | −3.4 | 0.00066 |
| ITENDIQIALDDAK_779.9_632.3 | 1.91E+00 | 6.78E+00 | 1.36E+00 | 1.4 | 0.16036 |

TABLE 3
Transitions selected by Cox lasso model

| Transition | coef | exp(coef) | se(coef) | z | Pr(>\|z\|) |
|---|---|---|---|---|---|
| Collection.Window.GA.in.Days | 0.0233 | 1.02357 | 0.00928 | 2.51 | 0.012 |
| AFTECCVVASQLR_770.87_574.3 | 1.07568 | 2.93198 | 0.84554 | 1.27 | 0.203 |
| ELLESYIDGR_597.8_710.3 | 1.3847 | 3.99365 | 0.70784 | 1.96 | 0.05 |
| ITLPDFTGDLR_624.34_920.4 | 0.814 | 2.25691 | 0.40652 | 2 | 0.045 |

TABLE 4
Area under the ROC (AUROC) curve for individual
analytes to discriminate pre-term birth subjects
from non-pre-term birth subjects. The 77 transitions
with the highest AUROC area are shown.

| Transition | AUROC |
|---|---|
| ELLESYIDGR_597.8_710.3 | 0.71 |
| AFTECCVVASQLR_770.87_574.3 | 0.70 |
| ITLPDFTGDLR_624.34_920.4 | 0.70 |
| IRPFFPQQ_516.79_661.4 | 0.68 |
| TDAPDLPEENQAR_728.34_613.3 | 0.67 |
| ITLPDFTGDLR_624.34_288.2 | 0.67 |
| ELLESYIDGR_597.8_839.4 | 0.67 |
| SFRPFVPR_335.86_635.3 | 0.67 |
| ETAASLLQAGYK_626.33_879.5 | 0.67 |
| TLLPVSKPEIR_418.26_288.2 | 0.66 |
| ETAASLLQAGYK_626.33_679.4 | 0.66 |
| SFRPFVPR_335.86_272.2 | 0.66 |
| LQGTLPVEAR_542.31_571.3 | 0.66 |
| VEPLYELVTATDFAYSSTVR_754.38_712.4 | 0.66 |
| DPDQTDGLGLSYLSSHIANVER_796.39_328.1 | 0.66 |
| VTGWGNLK_437.74_617.3 | 0.65 |
| ALQDQLVLVAAK_634.88_289.2 | 0.65 |
| EAQLPVIENK_570.82_329.1 | 0.65 |
| VRPQQLVK_484.31_609.3 | 0.65 |
| AFTECCVVASQLR_770.87_673.4 | 0.65 |
| YEFLNGR_449.72_293.1 | 0.65 |
| VGEYSLYIGR_578.8_871.5 | 0.64 |
| EAQLPVIENK_570.82_699.4 | 0.64 |
| TLLPVSKPEIR_418.26_514.3 | 0.64 |
| IEEIAAK_387.22_531.3 | 0.64 |
| LEQGENVFLQATDK_796.4_822.4 | 0.64 |
| LQGTLPVEAR_542.31_842.5 | 0.64 |
| FLQEQGHR_338.84_497.3 | 0.63 |
| ISLLLIESWLEPVR_834.49_371.2 | 0.63 |
| IITGLLEFEVYLEYLQNR_738.4_530.3 | 0.63 |
| LSSPAVITDK_515.79_743.4 | 0.63 |
| VRPQQLVK_484.31_722.4 | 0.63 |
| SLPVSDSVLSGFEQR_810.92_723.3 | 0.63 |
| VQEAHLTEDQIFYFPK_655.66_701.4 | 0.63 |
| NADYSYSVWK_616.78_333.2 | 0.63 |
| DAQYAPGYDK_564.25_813.4 | 0.62 |
| FQLPGQK_409.23_276.1 | 0.62 |
| TASDFITK_441.73_781.4 | 0.62 |
| YGLVTYATYPK_638.33_334.2 | 0.62 |
| GSFALSFPVESDVAPIAR_931.99_363.2 | 0.62 |
| TLLIANETLR_572.34_703.4 | 0.62 |
| VILGAHQEVNLEPHVQEIEVSR_832.78_860.4 | 0.62 |
| TATSEYQTFFNPR_781.37_386.2 | 0.62 |
| YEVQGEVFTKPQLWP_910.96_392.2 | 0.62 |
| DISEVVTPR_508.27_472.3 | 0.62 |
| IS-1_419.7_691.2 | 0.62 |
| GSFALSFPVESDVAPIAR_931.99_456.3 | 0.62 |
| YGFYTHVFR_397.2_421.3 | 0.62 |
| TLEAQLTPR_514.79_685.4 | 0.62 |
| YGFYTHVFR_397.2_659.4 | 0.62 |
| AVGYLITGYQR_620.84_737.4 | 0.61 |
| DPDQTDGLGLSYLSSHIANVER_796.39_456.2 | 0.61 |
| FNAVLTNPQGDYDTSTGK_964.46_262.1 | 0.61 |
| SPEQQETVLDGNLIIR_906.48_685.4 | 0.61 |
| ALNHLPLEYNSALYSR_620.99_538.3 | 0.61 |
| GGEIEGFR_432.71_508.3 | 0.61 |
| GIVEECCFR_585.26_900.3 | 0.61 |
| DAQYAPGYDK_564.25_315.1 | 0.61 |
| FAFNLYR_465.75_712.4 | 0.61 |
| YTTEIIK_434.25_603.4 | 0.61 |
| AVLTIDEK_444.76_605.3 | 0.61 |
| AITPPHPASQANIIFDITEGNLR_825.77_459.3 | 0.60 |
| EPGLCTWQSLR_673.83_790.4 | 0.60 |
| AVYEAVLR_460.76_587.4 | 0.60 |
| ALQDQLVLVAAK_634.88_956.6 | 0.60 |
| AWVAWR_394.71_531.3 | 0.60 |
| TNLESILSYPK_632.84_807.5 | 0.60 |
| HLSLLTTLSNR_418.91_376.2 | 0.60 |
| FTFTLHLETPKPSISSSNLNPR_829.44_787.4 | 0.60 |
| AVGYLITGYQR_620.84_523.3 | 0.60 |
| FQLPGQK_409.23_429.2 | 0.60 |
| YGLVTYATYPK_638.33_843.4 | 0.60 |
| TELRPGETLNVNFLLR_624.68_662.4 | 0.60 |
| LSSPAVITDK_515.79_830.5 | 0.60 |
| TATSEYQTFFNPR_781.37_272.2 | 0.60 |
| LPTAVVPLR_483.31_385.3 | 0.60 |
| APLTKPLK_289.86_260.2 | 0.60 |

TABLE 5
AUROCs for random forest, boosting, lasso, and logistic regression
models for a specific number of transitions permitted in the model,
as estimated by 100 rounds of bootstrap resampling.

| Number of | | | | |
|---|---|---|---|---|
| transitions | rf | boosting | logit | lasso |
| 1 | 0.59 | 0.67 | 0.64 | 0.69 |
| 2 | 0.66 | 0.70 | 0.63 | 0.68 |
| 3 | 0.69 | 0.70 | 0.58 | 0.71 |
| 4 | 0.68 | 0.72 | 0.58 | 0.71 |
| 5 | 0.73 | 0.71 | 0.58 | 0.68 |
| 6 | 0.72 | 0.72 | 0.56 | 0.68 |
| 7 | 0.74 | 0.70 | 0.60 | 0.67 |
| 8 | 0.73 | 0.72 | 0.62 | 0.67 |
| 9 | 0.72 | 0.72 | 0.60 | 0.67 |
| 10 | 0.74 | 0.71 | 0.62 | 0.66 |
| 11 | 0.73 | 0.69 | 0.58 | 0.67 |
| 12 | 0.73 | 0.69 | 0.59 | 0.66 |
| 13 | 0.74 | 0.71 | 0.57 | 0.66 |
| 14 | 0.73 | 0.70 | 0.57 | 0.65 |
| 15 | 0.72 | 0.70 | 0.55 | 0.64 |

TABLE 6
Top 15 transitions selected by each multivariate
method, ranked by importance for that method.

| | rf | boosting | lasso | logit |
|---|---|---|---|---|
| 1 | ELLESYIDGR_{—} | AFTECCVVASQL | AFTECCVVASQLR | ALQDQLVLVAAK_{—} |
| | 597.8_710.3 | R_770.87_574.3 | _770.87_574.3 | 634.88_289.2 |
| 2 | TATSEYQTFF | DPDQTDGLGLSY | ISLLLIESWLEPVR_{—} | AVLTIDEK_444.76_{—} |
| | NPR_781.37_38 | LSSHIANVER_796 | 834.49_371.2 | 605.3 |
| | 6.2 | .39_328.1 | | |
| 3 | ITLPDFTGDLR | ELLESYIDGR_597 | LPTAVVPLR_483.31 | Collection.Window.G |
| | _624.34_920.4 | .8_710.3 | _385.3 | A.in.Days |
| 4 | AFTECCVVAS | TATSEYQTFFNPR | ALQDQLVLVAAK_{—} | AHYDLR_387.7_566 |
| | QLR_770.87_57 | _781.37_386.2 | 634.88_289.2 | .3 |
| | 4.3 | | | |
| 5 | VEPLYELVTA | ITLPDFTGDLR_62 | ETAASLLQAGYK_{—} | AEAQAQYSAAVA |
| | TDFAYSSTVR | 4.34_920.4 | 626.33_679.4 | K_654.33_908.5 |
| | _754.38_712.4 | | | |
| 6 | GSFALSFPVES | GGEIEGFR_432.71 | IITGLLEFEVYLEYL | AEAQAQYSAAVA |
| | DVAPIAR_931. | _379.2 | QNR_738.4_530.3 | K_654.33_709.4 |
| | 99_363.2 | | | |
| 7 | VGEYSLYIGR_{—} | ALQDQLVLVAAK | ADSQAQLLLSTVV | ADSQAQLLLSTVV |
| | 578.8_871.5 | _634.88_289.2 | GVFTAPGLHLK_82 | GVFTAPGLHLK_82 |
| | | | 2.46_983.6 | 2.46_983.6 |
| 8 | SFRPFVPR_335 | VGEYSLYIGR_57 | SLPVSDSVLSGFEQ | AITPPHPASQANIIF |
| | .86_635.3 | 8.8_871.5 | R_810.92_723.3 | DITEGNLR_825.77_{—} |
| | | | | 459.3 |
| 9 | ALQDQLVLVA | VEPLYELVTATD | SFRPFVPR_335.86_{—} | ADSQAQLLLSTVV |
| | AK_634.88_289 | FAYSSTVR_754.3 | 272.2 | GVFTAPGLHLK_82 |
| | .2 | 8_712.4 | | 2.46_664.4 |
| 10 | EDTPNSVWEP | SPEQQETVLDGN | IIGGSDADIK_494.7 | AYSDLSR_406.2_37 |
| | AK_686.82_315 | LIIR_906.48_685.4 | 7_260.2 | 5.2 |
| | .2 | | | |
| 11 | YGFYTHVFR_{—} | YEFLNGR_449.72 | NADYSYSVWK_61 | DALSSVQESQVAQ |
| | 397.2_421.3 | _293.1 | 6.78_333.2 | QAR_572.96_672.4 |
| 12 | DPDQTDGLGL | LEQGENVFLQAT | GSFALSFPVESDVA | ANRPFLVFIR_411.5 |
| | SYLSSHIANVE | DK_796.4_822.4 | PIAR_931.99_456.3 | 8_435.3 |
| | R_796.39_328.1 | | | |
| 13 | LEQGENVFLQ | LQGTLPVEAR_54 | LSSPAVITDK_515.7 | DALSSVQESQVAQ |
| | ATDK_796.4_8 | 2.31_571.3 | 9_743.4 | QAR_572.96_502.3 |
| | 22.4 | | | |
| 14 | LQGTLPVEAR_{—} | ISLLLIESWLEPVR | ELPEHTVK_476.76_{—} | ALEQDLPVNIK_62 |
| | 542.31_571.3 | _834.49_371.2 | 347.2 | 0.35_570.4 |
| 15 | SFRPFVPR_335 | TASDFITK_441.73 | EAQLPVIENK_570. | AVLTIDEK_444.76_{—} |
| | .86_272.2 | _781.4 | 82_699.4 | 718.4 |

In yet another aspect, the invention provides kits for determining probability of preterm birth, wherein the kits can be used to detect N of the isolated biomarkers listed in Tables 1, 2, 3, 4, 6 and 7. For example, the kits can be used to detect one or more, two or more, or three of the isolated biomarkers selected from the group consisting of AFTECCVVASQLR, ELLESYIDGR, and ITLPDFTGDLR.

In another aspect, the kits can be used to detect one or more, two or more, three or more, four or more, five or more, six or more, seven or more, or eight of the isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G).

The kit can include one or more agents for detection of biomarkers, a container for holding a biological sample isolated from a pregnant female; and printed instructions for reacting agents with the biological sample or a portion of the biological sample to detect the presence or amount of the isolated biomarkers in the biological sample. The agents can be packaged in separate containers. The kit can further comprise one or more control reference samples and reagents for performing an immunoassay.

In one embodiment, the kit comprises agents for measuring the levels of at least N of the isolated biomarkers listed in Tables 1, 2, 3, 4, 6 and 7. The kit can include antibodies that specifically bind to these biomarkers, for example, the kit can contain at least one of an antibody that specifically binds to lipopolysaccharide-binding protein (LBP), an antibody that specifically binds to prothrombin (THRB), an antibody that specifically binds to complement component C5 (C5 or CO5), an antibody that specifically binds to plasminogen (PLMN), and an antibody that specifically binds to complement component C8 gamma chain (C8G or CO8G).

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining probability of preterm birth.

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The following examples are provided by way of illustration, not limitation.

### EXAMPLES

### Example 1

### Development of Sample Set for Discovery and Validation of Biomarkers for Preterm Birth

A standard protocol was developed governing conduct of the Proteomic Assessment of Preterm Risk (PAPR) clinical study. This protocol also specified that the samples and clinical information could be used to study other pregnancy complications. Specimens were obtained from women at 11 Internal Review Board (IRB) approved sites across the United States. After providing informed consent, serum and plasma samples were obtained, as well as pertinent information regarding the patient's demographic characteristics, past medical and pregnancy history, current pregnancy history and concurrent medications. Following delivery, data were collected relating to maternal and infant conditions and complications. Serum and plasma samples were processed according to a protocol that requires standardized refrigerated centrifugation, aliquoting of the samples into 0.5 ml 2-D bar-coded cryovials and subsequent freezing at −80° C.

Following delivery, preterm birth cases were individually reviewed to determine their status as either a spontaneous preterm birth or a medically indicated preterm birth. Only spontaneous preterm birth cases were used for this analysis. For discovery of biomarkers of preterm birth, 80 samples were analyzed in two gestational age groups: a) a late window composed of samples from 23-28 weeks of gestation which included 13 cases, 13 term controls matched within one week of sample collection and 14 term random controls, and, b) an early window composed of samples from 17-22 weeks of gestation included 15 cases, 15 term controls matched within one week of sample collection and 10 random term controls.

The samples were subsequently depleted of high abundance proteins using the Human 14 Multiple Affinity Removal System (MARS 14), which removes 14 of the most abundant proteins that are essentially uninformative with regard to the identification for disease-relevant changes in the serum proteome. To this end, equal volumes of each clinical or HGS sample were diluted with column buffer and filtered to remove precipitates. Filtered samples were depleted using a MARS-14 column (4.6×100 mm, Cat. #5188-6558, Agilent Technologies). Samples were chilled to 4° C. in the autosampler, the depletion column was run at room temperature, and collected fractions were kept at 4° C. until further analysis. The unbound fractions were collected for further analysis.

A second aliquot of each clinical serum sample and of each HGS was diluted into ammonium bicarbonate buffer and depleted of the 14 high and approximately 60 additional moderately abundant proteins using an IgY14-SuperMix (Sigma) hand-packed column, comprised of 10 mL of bulk material (50% slurry, Sigma). Shi et al., Methods, 56(2):246-53 (2012). Samples were chilled to 4° C. in the autosampler, the depletion column was run at room temperature, and collected fractions were kept at 4° C. until further analysis. The unbound fractions were collected for further analysis.

Depleted serum samples were denatured with trifluorethanol, reduced with dithiotreitol, alkylated using iodoacetamide, and then digested with trypsin at a 1:10 trypsin: protein ratio. Following trypsin digestion, samples were desalted on a C18 column, and the eluate lyophilized to dryness. The desalted samples were resolubilized in a reconstitution solution containing five internal standard peptides.

Depleted and trypsin digested samples were analyzed using a scheduled Multiple Reaction Monitoring method (sMRM). The peptides were separated on a 150 mm×0.32 mm Bio-Basic C18 column (ThermoFisher) at a flow rate of 5 μl/min using a Waters Nano Acquity UPLC and eluted using an acetonitrile gradient into a AB SCIEX QTRAP 5500 with a Turbo V source (AB SCIEX, Framingham, Mass.). The sMRM assay measured 1708 transitions that correspond to 854 peptides and 236 proteins. Chromatographic peaks were integrated using Rosetta Elucidator software (Ceiba Solutions).

Transitions were excluded from analysis, if their intensity area counts were less than 10000 and if they were missing in more than three samples per batch. Intensity area counts were log transformed and Mass Spectrometry run order trends and depletion batch effects were minimized using a regression analysis.

### Example 2

### Analysis I of Transitions to Identify Preterm Birth Biomarkers

The objective of these analyses was to examine the data collected in Example 1 to identify transitions and proteins that predict preterm birth. The specific analyses employed were (i) Cox time-to-event analyses and (ii) models with preterm birth as a binary categorical dependent variable. The dependent variable for all the Cox analyses was Gestational Age of time to event (where event is preterm birth). For the purpose of the Cox analyses, preterm birth subjects have the event on the day of birth. Term subjects are censored on the day of birth. Gestational age on the day of specimen collection is a covariate in all Cox analyses.

The assay data were previously adjusted for run order and depletion batch, and log transformed. Values for gestational age at time of sample collection were adjusted as follows. Transition values were regressed on gestational age at time of sample collection using only controls (non-pre-term subjects). The residuals from the regression were designated as adjusted values. The adjusted values were used in the models with pre-term birth as a binary categorical dependent variable. Unadjusted values were used in the Cox analyses.

Univariate Cox Proportional Hazards Analyses

Univariate Cox Proportional Hazards analyses was performed to predict Gestational Age at Birth, including Gestational age on the day of specimen collection as a covariate. Table 1 shows the transitions with p-values less than 0.05. Five proteins have multiple transitions among those with p-value less than 0.05: lipopolysaccharide-binding protein (LBP), prothrombin (THRB), complement component C5 (C5 or CO5), plasminogen (PLMN), and complement component C8 gamma chain (C8G or CO8G).

Multivariate Cox Proportional Hazards Analyses: Stepwise AIC Selection

Cox Proportional Hazards analyses was performed to predict Gestational Age at Birth, including Gestational age on the day of specimen collection as a covariate, using stepwise and lasso models for variable selection. These analyses include a total of n=80 subjects, with number of PTB events=28. The stepwise variable selection analysis used the Akaike Information Criterion (AIC) as the stopping criterion. Table 2 shows the transitions selected by the stepwise AIC analysis. The coefficient of determination (R²) for the stepwise AIC model is 0.86 (not corrected for multiple comparisons).

Multivariate Cox Proportional Hazards Analyses: Lasso Selection

Lasso variable selection was used as the second method of multivariate Cox Proportional Hazards analyses to predict Gestational Age at Birth, including Gestational age on the day of specimen collection as a covariate. This analysis uses a lambda penalty for lasso estimated by cross validation. Table 3 shows the results. The lasso variable selection method is considerably more stringent than the stepwise AIC, and selects only 3 transitions for the final model, representing 3 different proteins. These 3 proteins give the top 4 transitions from the univariate analysis; 2 of the top 4 univariate are from the same protein, and hence are not both selected by the lasso method. Lasso tends to select a relatively small number of variables with low mutual correlation. The coefficient of determination (R²) for the lasso model is 0.21 (not corrected for multiple comparisons).

Univariate AUROC Analysis of Preterm Birth as a Binary Categorical Dependent Variable

Univariate analyses was performed to discriminate pre-term subjects from non-pre-term subjects (pre-term as a binary categorical variable) as estimated by area under the receiver operating characteristic (AUROC) curve. These analyses use transition values adjusted for gestational age at time of sample collection, as described above. Table 4 shows the AUROC curve for the 77 transitions with the highest AUROC area of 0.6 or greater.

Multivariate Analysis of Preterm Birth as a Binary Categorical Dependent Variable

Multivariate analyses was performed to predict preterm birth as a binary categorical dependent variable, using random forest, boosting, lasso, and logistic regression models. Random forest and boosting models grow many classification trees. The trees vote on the assignment of each subject to one of the possible classes. The forest chooses the class with the most votes over all the trees.

For each of the four methods (random forest, boosting, lasso, and logistic regression) each method was allowed to select and rank its own best 15 transitions. We then built models with 1 to 15 transitions. Each method sequentially reduces the number of nodes from 15 to 1 independently. A recursive option was used to reduce the number nodes at each step: To determine which node to be removed, the nodes were ranked at each step based on their importance from a nested cross-validation procedure. The least important node was eliminated. The importance measures for lasso and logistic regression are z-values. For random forest and boosting, the variable importance was calculated from permuting out-of-bag data: for each tree, the classification error rate on the out-of-bag portion of the data was recorded; the error rate was then recalculated after permuting the values of each variable (i.e., transition); if the transition was in fact important, there would have been be a big difference between the two error rates; the difference between the two error rates were then averaged over all trees, and normalized by the standard deviation of the differences. The AUCs for these models are shown in Table 5 and in FIG. 1, as estimated by 100 rounds of bootstrap resampling. Table 6 shows the top 15 transitions selected by each multivariate method, ranked by importance for that method. These multivariate analyses suggest that models that combine 3 or more transitions give AUC greater than 0.7, as estimated by bootstrap.

In multivariate models, random forest (rf), boosting, and lasso models gave the best area under the AUROC curve. The following transitions were selected by these models, as significant in Cox univariate models, and/or having high univariate ROC's:

AFTECCVVASQLR_{—}770.87_{—}574.3

ELLESYIDGR_{—}597.8_{—}710.3

ITLPDFTGDLR_{—}624.34_{—}920.4

TDAPDLPEENQAR_{—}728.34_{—}613.3

SFRPFVPR_{—}335.86_{—}635.3

In summary, univariate and multivariate Cox analyses was performed using transitions to predict Gestational Age at Birth, including Gestational age on the day of specimen collection as a covariate. In the univariate Cox analysis, five proteins were identified that have multiple transitions among those with p-value less than 0.05: lipopolysaccharide-binding protein (LBP), prothrombin (THRB), complement component C5 (C5 or CO5), plasminogen (PLMN), and complement component C8 gamma chain (C8G or CO8G).

In multivariate Cox analyses, stepwise AIC variable analysis selects 24 transitions, while the lasso model selects 3 transitions, which include the 3 top proteins in the univariate analysis. Univariate (AUROC) and multivariate (random forest, boosting, lasso, and logistic regression) analyses were performed to predict pre-term birth as a binary categorical variable. Univariate analyses identified 63 analytes with AUROC of 0.6 or greater. Multivariate analyses suggest that models that combine 3 or more transitions give AUC greater than 0.7, as estimated by bootstrap.

### Example 3

### Study II to Identify and Confirm Preterm Birth Biomarkers

A further study was performed using essentially the same methods described in the preceding Examples unless noted below. In this study, 2 gestational aged matched controls were used for each case of 28 cases and 56 matched controls, all from the early gestational window only (17-22 weeks).

The samples were processed in 4 batches with each batch composed of 7 cases, 14 matched controls and 3 HGS controls. The LC-MS/MS analysis was performed with an Agilent Poroshell 120 EC-C18 column (2.1×50 mm, 2.7 μm) and an Agilent 6490 Triple Quadrapole mass spectrometer.

Data analysis included the use of conditional logistic regression where each matching triplet (case and 2 matched controls) was a stratum. The p-value reported in the table indicates whether there is a significant difference between cases and matched controls.

TABLE 7
Results of Study II

| Transition | Protein | Annotation | p-value |
|---|---|---|---|
| DFHINLFQVLPWLK | CFAB_HUMAN | Complement factor B | 0.006729512 |
| ITLPDFTGDLR | LBP_HUMAN | Lipopolysaccharide- | 0.012907017 |
| | | binding protein | |
| WWGGQPLWITATK | ENPP2_HUMAN | Ectonucleotide | 0.013346 |
| | | pyrophosphatase/ | |
| | | phosphodiesterase | |
| | | family member 2 | |
| TASDFITK | GELS_HUMAN | Gelsolin | 0.013841221 |
| AGLLRPDYALLGHR | PGRP2_HUMAN | N-acetylmuramoyl-L- | 0.014241979 |
| | | alanine amidase | |
| FLQEQGHR | CO8G_HUMAN | Complement | 0.014339596 |
| | | component C8 gamma | |
| | | chain | |
| FLNWIK | HABP2_HUMAN | Hyaluronan-binding | 0.014790418 |
| | | protein 2 | |
| EKPAGGIPVLGSLVNTVL | BPIB1_HUMAN | BPI fold-containing | 0.019027746 |
| K | | family B member 1 | |
| ITGFLKPGK | LBP_HUMAN | Lipopolysaccharide- | 0.019836986 |
| | | binding protein | |
| YGLVTYATYPK | CFAB_HUMAN | Complement factor B | 0.019927774 |
| SLLQPNK | CO8A_HUMAN | Complement | 0.020930939 |
| | | component C8 alpha | |
| | | chain | |
| DISEVVTPR | CFAB_HUMAN | Complement factor B | 0.021738046 |
| VQEAHLTEDQIFYFPK | CO8G_HUMAN | Complement | 0.021924548 |
| | | component C8 gamma | |
| | | chain | |
| SPELQAEAK | APOA2_HUMAN | Apolipoprotein A-II | 0.025944285 |
| TYLHTYESEI | ENPP2_HUMAN | Ectonucleotide | 0.026150038 |
| | | pyrophosphatase/ | |
| | | phosphodiesterase | |
| | | family member 2 | |
| DSPSVWAAVPGK | PROF1_HUMAN | Profilin-1 | 0.026607371 |
| HYINLITR | NPY_HUMAN | Pro-neuropeptide Y | 0.027432804 |
| SLPVSDSVLSGFEQR | CO8G_HUMAN | Complement | 0.029647857 |
| | | component C8 gamma | |
| | | chain | |
| IPGIFELGISSQSDR | CO8B_HUMAN | Complement | 0.030430996 |
| | | component C8 beta | |
| | | chain | |
| IQTHSTTYR | F13B_HUMAN | Coagulation factor | 0.031667664 |
| | | XIII B chain | |
| DGSPDVTTADIGANTPDA | PGRP2_HUMAN | N-acetylmuramoyl-L- | 0.034738338 |
| TK | | alanine amidase | |
| QLGLPGPPDVPDHAAYHP | ITIH4_HUMAN | Inter-alpha-trypsin | 0.043130591 |
| F | | inhibitor heavy | |
| | | chain H4 | |
| FPLGSYTIQNIVAGSTYLF | LCAP_HUMAN | Leucyl-cystinyl | 0.044698045 |
| STK | | aminopeptidase | |
| AHYDLR | FETUA_HUMAN | Alpha-2-HS- | 0.046259201 |
| | | glycoprotein | |
| SFRPFVPR | LBP_HUMAN | Lipopolysaccharide- | 0.047948847 |
| | | binding protein | |

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A panel of isolated biomarkers comprising N of the biomarkers listed in Tables 1, 2, 3, 4, 6 and 7.

2. The panel of claim 1, wherein N is a number selected from the group consisting of 2 to 24.

3. The panel of claim 2, wherein said panel comprises at least two of the isolated biomarkers selected from the group consisting of AFTECCVVASQLR (SEQ ID NO: 11, ELLESYIDGR (SEQ ID NO: 2), and ITLPDFTGDLR (SEQ ID NO: 3).

4. The panel of claim 2, wherein said panel comprises lipopolysaccharide-binding protein (LBP), prothrombin (THRB), complement component C5 (C5 or CO5), plasminogen (PLMN), and complement component C8 gamma chain (C8G or CO8G).

5. The panel of claim 2, wherein said panel comprises at least two isolated biomarkers selected from the group consisting of lipopolysaccharide-binding protein (LBP), prothrombin (THRB), complement component C5 (C5 or CO5), plasminogen (PLMN), and complement component C8 gamma chain (C8G or CO8G).

6. The panel of claim 2, wherein said panel comprises at least two isolated biomarkers selected from the group consisting of lipopolysaccharide-binding protein (LBP), prothrombin (THRB), complement component C5 (C5 or CO5), plasminogen (PLMN), complement component C8 gamma chain (C8G or CO8G), complement component 1, q subcomponent, B chain (C1QB), fibrinogen beta chain (FIBB or FIB), C-reactive protein (CRP), inter-alpha-trypsin inhibitor heavy chain H4 (ITIH4), chorionic somatomammotropin hormone (CSH), and angiotensinogen (ANG or ANGT).

7. A method of determining probability for preterm birth in a pregnant female, the method comprising detecting a measurable feature of each of N biomarkers selected from the biomarkers listed in Tables 1, 2, 3, 4, 6 and 7 in a biological sample obtained from said pregnant female, and analyzing said measurable feature to determine the probability for preterm birth in said pregnant female.

8. The method of claim 7, wherein said measurable feature comprises fragments or derivatives of each of said N biomarkers selected from the biomarkers listed in Tables 1, 2, 3, 4, 6 and 7.

9. The method of claim 7, wherein said detecting a measurable feature comprises quantifying an amount of each of N biomarkers selected from the biomarkers listed in Tables 1, 2, 3, 4, 6 and 7, combinations or portions and/or derivatives thereof in a biological sample obtained from said pregnant female.

10. The method of claim 9, further comprising calculating the probability for preterm birth in said pregnant female based on said quantified amount of each of N biomarkers selected from the biomarkers listed in Tables 1, 2, 3, 4, 6 and 7.

11. The method of claim 7, further comprising an initial step of providing a biomarker panel comprising N of the biomarkers listed in Tables 1, 2, 3, 4, 6 and 7.

12. The method of claim 7, further comprising an initial step of providing a biological sample from the pregnant female.

13. The method of claim 7, further comprising communicating said probability to a health care provider.

14. The method of claim 13, wherein said communication informs a subsequent treatment decision for said pregnant female.

15. The method of claim 7, wherein N is a number selected from the group consisting of 2 to 24.

16. The method of claim 15, wherein said N biomarkers comprise at least two of the isolated biomarkers selected from the group consisting of AFTECCVVASQLR (SEQ ID NO: 1), ELLESYIDGR (SEQ ID NO: 2), and ITLPDFTGDLR (SEQ ID NO: 3).

17. The method of claim 7, wherein said analysis comprises a use of a predictive model.

18. The method of claim 17, wherein said analysis comprises comparing said measurable feature with a reference feature.

19. The method of claim 18, wherein said analysis comprises using one or more selected from the group consisting of a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, and a combination thereof.

20. The method of claim 19, wherein said analysis comprises logistic regression.

21. The method of claim 7, wherein said probability is expressed as a risk score.

22. The method of claim 7, wherein the biological sample is selected from the group consisting of whole blood, plasma, and serum.

23. The method of claim 22, wherein the biological sample is serum.

24. The method of claim 7, wherein said quantifying comprises mass spectrometry (MS).

25. The method of claim 24, wherein said MS comprises liquid chromatography-mass spectrometry (LC-MS).

26. The method of claim 24, wherein said MS comprises multiple reaction monitoring (MRM) or selected reaction monitoring (SRM).

27. The method of claim 26, wherein said MRM (or SRM) comprises scheduled MRM (SRM).

28. The method of claim 7, wherein said quantifying comprises an assay that utilizes a capture agent.

29. The method of claim 28, wherein said capture agent is selected from the group consisting of and antibody, antibody fragment, nucleic acid-based protein binding reagent, small molecule or variant thereof.

30. The method of claim 28, wherein said assay is selected from the group consisting of enzyme immunoassay (EIA), enzyme-linked immunosorbent assay (ELISA), and radioimmunoassay (RIA).

31. The method of claim 30, wherein said quantifying further comprises mass spectrometry (MS).

32. The method of claim 31, wherein said quantifying comprises co-immunoprecitipation-mass spectrometry (co-IP MS).

33. The method of claim 7, further comprising detecting a measurable feature for one or more risk indicia.

34. The method of claim 33, wherein the one or more risk indicia are selected from the group consisting of history of previous low birth weight or preterm delivery, multiple 2nd trimester spontaneous abortion, prior first trimester induced abortion, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low prepregnancy weight/low body mass index, diabetes, hypertension, and urogenital infections.

35. A method of determining probability for preterm birth in a pregnant female, the method comprising: (a) quantifying in a biological sample obtained from said pregnant female an amount of each of N biomarkers selected from the biomarkers listed in Tables 1, 2, 3, 4, 6 and 7; (b) multiplying said amount by a predetermined coefficient, (c) determining the probability for preterm birth in said pregnant female comprising adding said individual products to obtain a total risk score that corresponds to said probability.


SEQUENCE LISTING (95 sequences)

SEQ ID NO: 1 - protein, 13 aa, Homo sapiens
AFTECCVVASQLR

SEQ ID NO: 2 - protein, 10 aa, Homo sapiens
ELLESYIDGR

SEQ ID NO: 3 - protein, 11 aa, Homo sapiens
ITLPDFTGDLR

SEQ ID NO: 4 - protein, 13 aa, Homo sapiens
TDAPDLPEENQAR

SEQ ID NO: 5 - protein, 8 aa, Homo sapiens
SFRPFVPR

SEQ ID NO: 6 - protein, 14 aa, Homo sapiens
LEQGENVFLQATDK

SEQ ID NO: 7 - protein, 12 aa, Homo sapiens
ETAASLLQAGYK

SEQ ID NO: 8 - protein, 8 aa, Homo sapiens
VTGWGNLK

SEQ ID NO: 9 - protein, 10 aa, Homo sapiens
EAQLPVIENK

SEQ ID NO: 10 - protein, 8 aa, Homo sapiens
FLQEQGHR

SEQ ID NO: 11 - protein, 8 aa, Homo sapiens
IRPFFPQQ

SEQ ID NO: 12 - protein, 11 aa, Homo sapiens
TLLPVSKPEIR

SEQ ID NO: 13 - protein, 10 aa, Homo sapiens
LSSPAVITDK

SEQ ID NO: 14 - protein, 15 aa, Homo sapiens
YEVQGEVFTKPQLWP

SEQ ID NO: 15 - protein, 10 aa, Homo sapiens
LQGTLPVEAR

SEQ ID NO: 16 - protein, 8 aa, Homo sapiens
VRPQQLVK

SEQ ID NO: 17 - protein, 7 aa, Homo sapiens
IEEIAAK

SEQ ID NO: 18 - protein, 16 aa, Homo sapiens
VQEAHLTEDQIFYFPK

SEQ ID NO: 19 - protein, 14 aa, Homo sapiens
ISLLLIESWLEPVR

SEQ ID NO: 20 - protein, 12 aa, Homo sapiens
ALQDQLVLVAAK

SEQ ID NO: 21 - protein, 7 aa, Homo sapiens
YEFLNGR

SEQ ID NO: 22 - protein, 18 aa, Homo sapiens
TPSAAYLWVGTGASEAEK

SEQ ID NO: 23 - protein, 13 aa, Homo sapiens
TATSEYQTFFNPR

SEQ ID NO: 24 - protein, 8 aa, Homo sapiens
TASDFITK

SEQ ID NO: 25 - protein, 18 aa, Homo sapiens
IITGLLEFEVYLEYLQNR

SEQ ID NO: 26 - protein, 10 aa, Homo sapiens
IIGGSDADIK

SEQ ID NO: 27 - protein, 7 aa, Homo sapiens
YTTEIIK

SEQ ID NO: 28 - protein, 12 aa, Homo sapiens
EDTPNSVWEPAK

SEQ ID NO: 29 - protein, 8 aa, Homo sapiens
LYYGDDEK

SEQ ID NO: 30 - protein, 8 aa, Homo sapiens
GGEIEGFR

SEQ ID NO: 31 - protein, 20 aa, Homo sapiens
DGSPDVTTADIGANTPDATK

SEQ ID NO: 32 - protein, 6 aa, Homo sapiens
VEIDTK

SEQ ID NO: 33 - protein, 8 aa, Homo sapiens
AVLTIDEK

SEQ ID NO: 34 - protein, 7 aa, Homo sapiens
FSVVYAK

SEQ ID NO: 35 - protein, 7 aa, Homo sapiens
YYLQGAK

SEQ ID NO: 36 - protein, 13 aa, Homo sapiens
EENFYVDETTVVK

SEQ ID NO: 37 - protein, 9 aa, Homo sapiens
YGFYTHVFR

SEQ ID NO: 38 - protein, 10 aa, Homo sapiens
HTLNQIDEVK

SEQ ID NO: 39 - protein, 12 aa, Homo sapiens
AFIQLWAFDAVK

SEQ ID NO: 40 - protein, 8 aa, Homo sapiens
SGFSFGFK

SEQ ID NO: 41 - protein, 11 aa, Homo sapiens
GWVTDGFSSLK

SEQ ID NO: 42 - protein, 14 aa, Homo sapiens
ITENDIQIALDDAK

SEQ ID NO: 43 - protein, 20 aa, Homo sapiens
VEPLYELVTATDFAYSSTVR

SEQ ID NO: 44 - protein, 22 aa, Homo sapiens
DPDQTDGLGLSYLSSHIANVER

SEQ ID NO: 45 - protein, 10 aa, Homo sapiens
VGEYSLYIGR

SEQ ID NO: 46 - protein, 15 aa, Homo sapiens
SLPVSDSVLSGFEQR

SEQ ID NO: 47 - protein, 10 aa, Homo sapiens
NADYSYSVWK

SEQ ID NO: 48 - protein, 10 aa, Homo sapiens
DAQYAPGYDK

SEQ ID NO: 49 - protein, 7 aa, Homo sapiens
FQLPGQK

SEQ ID NO: 50 - protein, 11 aa, Homo sapiens
YGLVTYATYPK

SEQ ID NO: 51 - protein, 18 aa, Homo sapiens
GSFALSFPVESDVAPIAR

SEQ ID NO: 52 - protein, 10 aa, Homo sapiens
TLLIANETLR

SEQ ID NO: 53 - protein, 22 aa, Homo sapiens
VILGAHQEVNLEPHVQEIEVSR

SEQ ID NO: 54 - protein, 9 aa, Homo sapiens
DISEVVTPR

SEQ ID NO: 55 - protein, 9 aa, Homo sapiens
TLEAQLTPR

SEQ ID NO: 56 - protein, 11 aa, Homo sapiens
AVGYLITGYQR

SEQ ID NO: 57 - protein, 18 aa, Homo sapiens
FNAVLTNPQGDYDTSTGK

SEQ ID NO: 58 - protein, 16 aa, Homo sapiens
SPEQQETVLDGNLIIR

SEQ ID NO: 59 - protein, 16 aa, Homo sapiens
ALNHLPLEYNSALYSR

SEQ ID NO: 60 - protein, 9 aa, Homo sapiens
GIVEECCFR

SEQ ID NO: 61 - protein, 7 aa, Homo sapiens
FAFNLYR

SEQ ID NO: 62 - protein, 23 aa, Homo sapiens
AITPPHPASQANIIFDITEGNLR

SEQ ID NO: 63 - protein, 11 aa, Homo sapiens
EPGLCTWQSLR

SEQ ID NO: 64 - protein, 8 aa, Homo sapiens
AVYEAVLR

SEQ ID NO: 65 - protein, 6 aa, Homo sapiens
AWVAWR

SEQ ID NO: 66 - protein, 11 aa, Homo sapiens
TNLESILSYPK

SEQ ID NO: 67 - protein, 11 aa, Homo sapiens
HLSLLTTLSNR

SEQ ID NO: 68 - protein, 22 aa, Homo sapiens
FTFTLHLETPKPSISSSNLNPR

SEQ ID NO: 69 - protein, 16 aa, Homo sapiens
TELRPGETLNVNFLLR

SEQ ID NO: 70 - protein, 9 aa, Homo sapiens
LPTAVVPLR

SEQ ID NO: 71 - protein, 8 aa, Homo sapiens
APLTKPLK

SEQ ID NO: 72 - protein, 6 aa, Homo sapiens
AHYDLR

SEQ ID NO: 73 - protein, 13 aa, Homo sapiens
AEAQAQYSAAVAK

SEQ ID NO: 74 - protein, 24 aa, Homo sapiens
ADSQAQLLLSTVVGVFTAPGLHLK

SEQ ID NO: 75 - protein, 7 aa, Homo sapiens
AYSDLSR

SEQ ID NO: 76 - protein, 16 aa, Homo sapiens
DALSSVQESQVAQQAR

SEQ ID NO: 77 - protein, 10 aa, Homo sapiens
ANRPFLVFIR

SEQ ID NO: 78 - protein, 8 aa, Homo sapiens
ELPEHTVK

SEQ ID NO: 79 - protein, 11 aa, Homo sapiens
ALEQDLPVNIK

SEQ ID NO: 80 - protein, 8 aa, Homo sapiens
AVLTIDEK

SEQ ID NO: 81 - protein, 14 aa, Homo sapiens
DFHINLFQVLPWLK

SEQ ID NO: 82 - protein, 13 aa, Homo sapiens
WWGGQPLWITATK

SEQ ID NO: 83 - protein, 14 aa, Homo sapiens
AGLLRPDYALLGHR

SEQ ID NO: 84 - protein, 6 aa, Homo sapiens
FLNWIK

SEQ ID NO: 85 - protein, 19 aa, Homo sapiens
EKPAGGIPVLGSLVNTVLK

SEQ ID NO: 86 - protein, 9 aa, Homo sapiens
ITGFLKPGK

SEQ ID NO: 87 - protein, 7 aa, Homo sapiens
SLLQPNK

SEQ ID NO: 88 - protein, 9 aa, Homo sapiens
SPELQAEAK

SEQ ID NO: 89 - protein, 10 aa, Homo sapiens
TYLHTYESEI

SEQ ID NO: 90 - protein, 12 aa, Homo sapiens
DSPSVWAAVPGK

SEQ ID NO: 91 - protein, 8 aa, Homo sapiens
HYINLITR

SEQ ID NO: 92 - protein, 15 aa, Homo sapiens
IPGIFELGISSQSDR

SEQ ID NO: 93 - protein, 9 aa, Homo sapiens
IQTHSTTYR

SEQ ID NO: 94 - protein, 19 aa, Homo sapiens
QLGLPGPPDVPDHAAYHPF

SEQ ID NO: 95 - protein, 22 aa, Homo sapiens
FPLGSYTIQNIVAGSTYLFSTK